(19) United States
(12) Patent Application Publication
(10) Pub. No.: US 2022/0228277 A1
(43) Pub. Date: Jul. 21, 2022

(54) COMPACT GENERATOR FOR GENERATING STERILIZING MATERIALS

(71) Applicant: Lih-Ren SHIUE, Hsinchu (TW)

(72) Inventors: Lih-Ren SHIUE, Hsinchu (TW); Zhen-Hua FEI, Suzhou (CN)

(21) Appl. No.: 17/154,804

(22) Filed: Jan. 21, 2021

(51) Int. Cl. C25B 9/65; C25B 1/13; C25B 11/047; A61L 2/20; A01N 59/00

(52) U.S. Cl. CPC C25B 9/65; C25B 1/13; C25B 11/047; A61L 2101/02; A61L 2/208; A01N 59/00; A61L 2/202

(57) ABSTRACT

The compact devices with built-in power can be constructed for producing disinfectants that can impart hygiene and sterilization to the device users. The disinfectants may include ozone (O₃), hydrogen peroxide (H₂O₂), peroxone (H₂O₃), singlet oxygen (O), hydroxy radical (.OH) and hydroperoxyl radical (HO₂.). In the electrolysis, the anode generates O₂ and O₃, whereas the cathode products, namely, either hydrogen gas (H₂) or H₂O₂, is dependent on the cathode materials utilized. When SS304 is used as the cathode, H₂ will be generated. On the other hand, H₂O₂ is formed on using cobalt oxide plated on carbon nanofilm coated Ti (Co₃O₄-CNF/Ti) as cathode. On using the latter, O₃ & H₂O₂ can be electrocatalytically cogenerated. When H₂O₂ mixes with O₃, H₂O₃ will be formed, so are .OH and HO₂.. O₃ and H₂O₂ can not only contribute O₂ to help human beings' breathing, they can impart human beings good health as well.

### BACKGROUND OF THE INVENTION

### 1. Field of the Invention

This invention relates to electrocatalytic generation of ozone (O₃), or electrocatalytic cogeneration of O₃ and hydrogen peroxide (H₂O₂) for sanitation and disinfection. More specifically, the invention relates to compact generators for producing O₃ or O₃/H₂O₂ by directly electrolyzing tap water using DC 5 volt. The disinfectant-containing water can be applied to the desired surfaces for sterilization.

### 2. Description of the Prior Art

In the COVID 19 pandemic, sodium hypochlorite (NaClO), commonly known as bleach, and ethanol are called for sterilization against the virus. The said disinfectants have slow bactericidal activity, and they cost money to procure. Compared to NaClO and C₂H₅OH, O₃ and H₂O₂ are benign, more potent, and quicker on killing microbes without residues. Most importantly, O₃ and H₂O₂ can be freshly made at home and other places as needs occurred. This patent presents an affordable way to produce O₃/H₂O₂ using tap water at low power consumption and user friendly. As far as the application and safety of O₃/H₂O₂ on disinfection is concerned, there are tons of literature references available, which is accessible in many searching engines. Only a few selected examples are cited here.

O₃-water has been investigated in a viral inactivation as seen in U.S. Patent Application No. U.S. 2005/0051497, coronavirus is included therein. An ozone hemofildiafiltration device is used for safe injection of O₃ into human body to kill cells of bacteria and virus in the blood as seen in US Patent Application No. U.S. 2003/0073945. Besides sterilization (U.S. Pat. Nos. 7,323,149 and 7,790,103), O₃ turns into O₂ at the end of its natural life in 20 to 30 minutes environment dependent. This feature grants O₃ a unique role in blood oxygen therapy by enhancing the blood oxygen level. Consequently, ozone is utilized in the cares of leukemia (U.S. Pat. No. 6,399,664), artificial lung (U.S. Pat. No. 7,498,275), acute ischemic brain stroke (US 2010/0318014), wounds (U.S. Pat. Nos. 8,597,689 and 9,687,503), and kidney disease (U.S. Pat. No. 10,335,538). The aforesaid uses of O₃ all require the disinfectant in tiny bubble forms.

Although the oxidation potential of H₂O₂ (1.78V) is lower than that of O₃ (2.07V), meaning, O₃ is superior to H₂O₂ on destructing microbes. However, H₂O₂ contains more O₂ and H₂O₂ works much longer than O₃, which is shown by the fact that a sealed H₂O₂—H₂O solution decays only 10% in a year. In reality, the history of applying H₂O₂ to medication is much earlier and wider than O₃. In 1920, H₂O₂ was injected to treat patients during an epidemic of viral pneumonia. Many promoters of blood oxygen therapy endorsed the ideas of Otto Warburg, a German physiologist, medical doctor, and Nobel laureate of 1931. He claimed that cancer cells grow better under low blood oxygen levels, and cancer could be cured by raising the O₂ level. The key on utilizing H₂O₂ for oxygenation mainly resides on the timely availability of fresh and pure H₂O₂ at the right dosages. This patent presents a device that may meet the demands.

### SUMMARY OF THE INVENTION

The present invention is to provide a compact generator for generating sterilizing materials. The compact generator includes an electrolysis module configured for electrolyzing water to generate at least one sterilizing material. The electrolysis module further includes an anode and a cathode. The anode is configured to generate oxygen (O₂) and ozone (O₃) during electrolyzing water. The cathode is configured on one side of the anode. The cathode is configured to produce hydrogen peroxide (H₂O₂) by reducing O₂ generated from the anode during electrolyzing water. The material of the anode is Sb,Ni—SnO₂/Ti. the material of the cathode is Co₃O₄-CNF/Ti, and the CNF is conductive carbon nanofilm. O₃ and H₂O₂ are cogenerated to form the at least one sterilizing material, and the sterilizing material includes O₃, peroxone (H₂O₃), hydroxyl radical (.OH) and hydroperoxyl radical (HO₂.).

Wherein, the precursor for the main body of anode catalysts, tin oxide (SnO₂), is tin oxalate (SnC₂O₄).

Furthermore, the tin oxalate is formed by oxalic acid and Tin(II) salt. The Tin(II) salt may be selected from the group of Dibutyltin dichloride, Dibutyltin maleate, Tin(II) acetate, Tin(II) bromide, Tin(II) chloride, Tin(II) 2-hexylhexanoate, Tin(II) fluoride, Tin(II) fluoroborate, Tin(II) iodide, Tin(II) pyrophosphate, Tin(II) sulfate and Tin(II) sulfide.

Wherein, the compact generator includes at least one supercapacitor and a power source. The supercapacitor is coupled to the electrolysis module and the power source. The supercapacitor is configured to discharge and store power from the power source and provide power to the electrolysis module for electrolyzing water.

Wherein, the supercapacitor includes a plurality of bipolar electrodes, and the bipolar electrodes are stacked in series.

Furthermore, the end bipolar electrodes of the bipolar electrode stack are connected to a power source and to receive the polarity from the poles of the power source.

Wherein, the two end electrodes of the supercapacitor may perform direct current (DC) or alternating current (AC) property via polarity reversal modulated by a controller.

Wherein, at least two identical supercapacitors can form a power module linked to a rechargeable battery for continuous delivery of power. The supercapacitors include a first supercapacitor and a second supercapacitor. The first supercapacitor and second supercapacitor perform reciprocal charging and discharging swing to meet all power demands with high efficiency of energy utilization.

Wherein, the supercapacitor can harvest power, using reverse charging, of return current from loads to the negative poles of power source through the reverse sides of electrodes of the supercapacitor.

In one embodiment, the compact generator includes a standard universal serial bus (USB) connector connected to the supercapacitor and the power source. The supercapacitor storing power through the standard USB connector.

In one embodiment, the supercapacitor may store power by wireless charging as well.

For facile production of O₃, as well as for making H₂O₂ simultaneously, the present invention provides a compact generator comprised of 4 subsystems for the said goals. The 4 subsystems include: (1 an anode to form O₂ and O₃ from water electrolysis, (2) 2 kinds of cathode to support the operation of anode by forming H₂, or by reducing O₂ from anode to H₂O₂, (3) a built-in energy storing arrays that can be charged with easy access everywhere, and (4) a smart IC circuitry that controls charging-discharging swing, for constant delivery of power at the desired level and a high efficiency of energy utilization.

Following are the four subsystems:

### (1) Electrocatalytic Generation of O₂ and O₃

The two electrodes for electrolyzing water to generate O₂—O₃ mixture employ antimony, nickel-doped tin oxide deposited on titanium metal (Sb,Ni—SnO₂/Ti) as anode, which is coupled with H₂ evolution by a food-grade AISI (American Iron and Steel Institute) 304 or SS304 as cathode. There is no membrane or separator disposed between anode and cathode, wherein the electrodes are merely fixed at a selected gap. Tap water or other clean water serves as the electrolysis medium without the use of additives. This means that water is the source of ozone. Gaseous products on both of anode and cathode are first formed in sub-micron sizes, then, most of the gases will grow into bigger sizes that escape to the nearby open spaces. Only a small amount of the gases produced, about 0.3% or less of the total gas weight, can dissolve in water. Nevertheless, the concentration of gases in water may be increased by cumulation, pressurization or cooling.

### (2) Electrocatalytic Co-Generation of O₃ and H₂O₂

Same anode and electrode configuration as (1), but the cathode is replaced by a carbon nanofilm doped with cobalt oxide, which is thermally grown on titanium metal (Co₃O₄-CNT/Ti). The said cathode produces no H₂, but it yields H₂O₂ by reducing the products of anode, that is, O₂ and proton (H⁺). The said products quickly diffuse to the cathode, thereon O₂ and H⁺ are reduced to H₂O₂ by cobalt oxide catalyst via a 2-electron mechanism. Some O₃ may mix with H₂O₂ to form peroxone (H₂O₃), hydroxy radical (.OH) and hydroperoxyl radical (HO₂.). The radicals are more potent than either O₃ or H₂O₂. Thus, the efficacy of sterilization is prolonged and the odor of O₃ is avoided.

### (3) Built-In Energy Storing Arrays

Supercapacitor alone, or an integration of the capacitor with a secondary battery can constitute an on-board energy storing arrays for the compact generators. In the integration, the supercapacitor serves as an energy buffer for the battery, wherein charging and discharging will begin with the capacitor. In the charging mode, the charged energy is first stored in the supercapacitor due to the quick charging rates and the large acceptance of charging currents of the capacitor. Once the energy stored in the capacitor is full, it will be transferred to the battery for long-term storage and lower energy loss based on the current leakage of battery is lower. Thereby, the supercapacitor utilizes the battery as energy backup for meeting all power demands.

Under the management of a smart circuitry to be described below, battery can always charge the capacitor only by low charging rates. When the disinfection devices require power, all needs will be provided by the supercapacitor using the advantages of quick discharging rates and high power-density of the capacitor. Regardless of the power level of demands, the supercapacitor can always respond in high rates and in real-time speeds, which cause no hurt to the capacitor. Should the battery be used as demanding as the capacitor, the lifetime of battery will be seriously compromised. As an energy buffer, the supercapacitor is performing “load leveling” for the battery by preventing it from delivering large powers. Hence, the battery can always discharge at very low rates so that its energy is preserved and lifetime is prolonged.

### (4) Smart Control Circuitry

Power is the driving force for the compact generators of this invention to produce the disinfectants of reactive oxygen species (ROS), namely, O₃, H₂O₂, H₂O₃, .OH and HO₂.. The energy refill and energy utilization of supercapacitor and the secondary battery are managed in the highest possible efficiency via the following innovative ways:

a. The energy-storage arrays are charged through a standard USB (Universal Serial Bus) Type A charger, which is widely utilized in cellular, laptops, desktop, tablets and other 3C merchandises.

b. Charging-Discharging Swing platform—Using 2 or more identical groups of supercapacitors in conjunction with 2° batteries for power provision, the energy content in each group of capacitors is controlled to release only their effective energy, which is the energy under the first ⅓ portion of the rated voltages. When the 1st group has discharged as scheduled, it will go into the charging mode, and the 2nd group immediately assumes the role of discharging. In the next cycle, the same sequence of reciprocal charging and discharging is repeated. The two groups of supercapacitors using the energy replenished by batteries to constantly deliver the required power in full until the demand is fulfilled. In the CD swing, the supercapacitors of this patent can incessantly provide power, which does not occur in the prevailing supercapacitors. An ASIC controller can direct the operation of CD Swing, wherein the level of output power can be decided by adjusting the switching frequency.

c. Reverse charging platform—The supercapacitors of this patent application are made of bipolar electrodes stacked juxtaposedly. Both sides of a bipolar electrode are coated with the same electrically active materials. While one side of the bipolar electrode is discharging, the reverse side is charged concurrently. It is the returning current, which returns from a load to the negative pole of a power source, provides the energy for harvest. On its way back, the return current charges the reverse side of a bipolar electrode. Hence, as one side of a bipolar electrode is discharging, the reverse side is being charged simultaneously at the same rate. Eventually, the reverse side of a bipolar electrode will develop a higher voltage but in negative polarity (−V) than the discharging side that is losing its positive voltage. In order to utilize the energy newly attained on the reverse side, the polarity of supercapacitor's leads must be reversed. By polarity reversal, energy can come from the side with a higher level of voltage. Reverse charging belongs to one kind of energy harvest, which imparts the energy utilization of supercapacitor high efficiency.

### BRIEF DESCRIPTION OF THE APPENDED DRAWINGS

The present invention is best understood by reference to the embodiments described in the subsequent sections accompanied with the following drawings.

FIG. 1 is a schematic diagram of a pocket-size ozone pen, which is portable and battery driven.

FIG. 2A to FIG. 2D are schematic diagrams of 4 kinds of compact portable devices for generating disinfectants.

FIG. 3 is a schematic diagram of an embodiment of a compact generator according to the present invention.

FIG. 4 is an exploded-view drawing of the compact generator in FIG. 3.

FIG. 5 is a logistic function blocks for providing a constant current for producing O₃ and H₂O₂.

### DETAILED DESCRIPTION OF THE INVENTION

A detailed description of the hereinafter described embodiments of the disclosed apparatus and method are presented herein by way of exemplification with reference to the Figures. Although certain embodiments are shown and described in detail, it should be understood that various changes and modifications may be made without departing from the scope of the appended claims. The scope of the present invention will in no way be limited to the number of constituting components, the materials thereof, the shapes thereof, the relative arrangement thereof, etc., and are disclosed simply as an example of embodiments of the present invention.

Human beings are living in an environment filled with bacteria, pathogens, and viruses. We need a handy and effective means for guarding ourselves against the harmful and invisible microbes. Ozone (O₃) and hydrogen peroxide (H₂O₂) are two well-known and powerful disinfectants for killing the invisible disease-inducers without byproducts produced. Most importantly, the two disinfectants can be generated under control and at a low cost whenever and wherever they are needed.

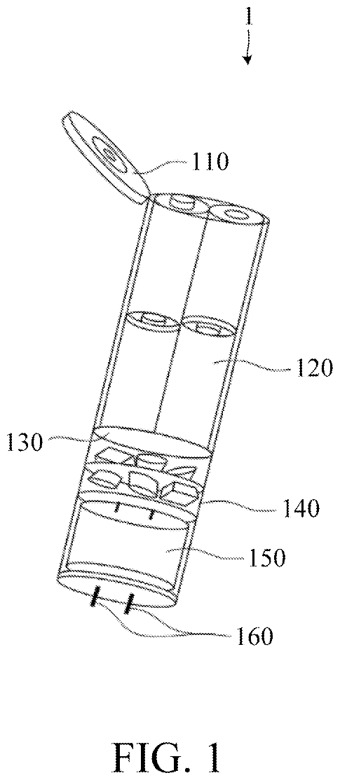

Please refer to FIG. 1. FIG. 1 is a schematic diagram of a pocket-size ozone pen 1, which is portable and battery driven. One of the inventors of the current patent application has proposed an ozone pen prototype more than a decade ago, which is described in US 2008/0181832. The original design of the O₃ pen is shown in FIG. 1, wherein the pen body has a lid 110, which covers contents including a battery compartment 120, rechargeable batteries 130, IC board 140, supercapacitor 150, as well as a pair of electrodes 160. At the time of filing, platinum plated on titanium (Pt/Ti) was used as anode, and SS304 served as cathode for the pen. In the afterwards development, a better anode material has been identified and implemented in this patent. Table 1 lists a group of the mostly tested anodic materials for making O₃ from water, with a material that can yield the O₃ gas from air is also included for comparison.

TABLE 1
Comparison of Methods for Artificial Generation of Ozone Sources

| | | Anodic | | | Power | |
|---|---|---|---|---|---|---|
| | | Materials/Gaseous | Operation | | Consumed | |
| # | By | Method | Volt (V) | T (° C.) | (kWh/kg O₃) | Efficiency (%) |
| 1 | H₂O | Pt | 5 | 20-25 | 20-40 | 4 |
| 2 | | Sb,Ni—SnO₂ | 5-24 | 20-25 | 18-30 | 24-54 |
| 3 | | β-PbO₂ | 3-5 | 5-20 | 65-54 | 13-50 |
| 4 | | Boron-doped | 30 | 5 | 130 | 10 |
| | | diamond (BDD) | | | | |
| 5 | Air or O₂ | Dielectrics | 3,000 | 20-25 | 20-30 | 4 |
| | | (Corona Discharge) | | | | |

In Table 1, the first four kinds of catalyst are employed in a technique named as electrocatalytic ozone (EO₃), wherein water is electrolyzed. Item #5 employs air or pure oxygen as the source of O₃. Catalyst is the heart of EO₃ from the perspectives of performance and lifetime. Among the EO₃ anodes, Sb and Ni doped tin oxide (Sb,Ni—SnO₂) is the best choice of catalyst due to its low cost, high performance, high CP value, easy implementation and long service life. Nevertheless, it is not an easy job to realize the good merits of (Sb,Ni—SnO₂) for commercial uses viably. In the metal contents of the said catalyst, Sn is the majority at over 94% by weight, or over 96% by mole ratio, whereas Sb is secondly abundant at 3-5%, and Ni is in a “tiny” amount (0.2. atomic % to Sn). Though at almost trace level, the catalyst will produce no O₃ without Ni. Not only the presence of Ni is a pivotal role, the distribution of Ni on the surface of catalyst also determines the service life of anode for the O₃ generator.

By using SnO₂ lattice as a platform, Sb and Ni present themselves in Sb₂O₅ and Ni₂O₃, respectively, in the adjacent vacancies of SnO₂ lattice. While Sb is doped to impart SnO₂ conductivity as in antimony-doped tin oxide (ATO), Ni drives the oxygen evolution potential of SnO₂ to a level where O₃ will be also formed. Together, Sb₅⁺ and Ni₃⁺ serve as an active site for the generation of O₃. Since SnO₂ lattice is the foundation for the Sb₅⁺—Ni₃⁺ active sites of O₃ generation, the said lattice must be built in the right order and clean structure. It is the precursor of SnO₂ that principally determines the quality of Sb,Ni—SnO₂ catalyst. When the SnO₂ lattice is compromised by contaminants, the Sb₅^{+—Ni}₃⁺ active sites would have no chance to build up adequately. In our long terms (decade-long) efforts of development towards EO₃ commercialization, we have verified that stannous oxalate or Ti(II) oxalate, SnC₂O₄, is the most suitable precursor for SnO₂ towards O₃ generation. Compared to the mostly chosen precursor of SnO₂ in the literatures, namely SnCl₄, SnC₂O₄ has the advantages of higher melting point (MP), 280° C. vs. 56° C., and free of chloride. Low MP causes tremendous loss of Sn at the drying stage of the coating solution if SnCl₄ is used. Also, chloride ion (Cl⁻) of SnCl₄ will form HCl during the fabrication of catalyst, which is hazardous to operators and equipment.

SnC₂O₄ can be facilely synthesized via metathesis reaction between a Sn(II) salt and oxalic acid (H₂C₂O₄) as described in equation (1)

SnX₂ (or SnY)+H₂C₂O₄→SnC₂O₄↓+2HX (or H₂Y) (1)

In Equation (1), X is a singly charged anion (X⁻¹) and Y has a charge count of 2 (Y⁻²). As soon as the two reactants are mixed, SnC₂O₄ will spontaneously and quickly form as a white precipitate. Then, the precipitate is filtered, rinsed to eliminate the acid contaminant, HX or H₂Y, finally the wet particles are dried before use. Table 2 lists a group of Sn(II) salts that may be employed as the precursor of SnC₂O₄.

TABLE 2
Tin(II) Salts for Synthesizing SnC₂O₄

| | | | CAS |
|---|---|---|---|
| # | Ti (II) Salts | Formulas | Number |
| 1 | Dibutyltin | (CH₃C₃H₆)₂SnCl₂ | 683-18-1 |
| | dichloride | | |
| 2 | Dibutyltin maleate | (CH₃C₃H₆)₂Sn[C₂H₂(COO)₂] | 78-04-6 |
| 3 | Tin(II) acetate | Sn(CH₃COO)₂ | 638-39-1 |
| 4 | Tin(II) bromide | SnBr₂ | 10031-24-0 |
| 5 | Tin(II) chloride | SnCl₂ | 7772-99-8 |
| 6 | Tin(II) | Sn[CH₃(CH₂)₃CH(C₂H₅)CO₂]₂ | 301-10-0 |
| | 2-ethylhexanoate | | |
| 7 | Tin(II) fluoride | SnF₂ | 7783-47-3 |
| 8 | Tin(II) fluoroborate | Sn(BF₄)₂ | 13814-97-6 |
| 9 | Tin(II) iodide | SnI₂ | 10294-70-9 |
| 10 | Tin(II) | Sn₂P₂O₇ | 15578-26-4 |
| | pyrophosphate | | |
| 11 | Tin(II) sulfate | SnSO₄ | 7488-55-3 |
| 12 | Tin(II) sulfide | SnS | 1314-95-0 |

So long as the two reactants of Equation (1) can be purified successfully, a high-quality SnC₂O₄ may be prepared. The reactants may be an industrial grade, which will produce a significant advantage in cost. For example, pound for pound, oxalic acid of the reagent grade is at least 28 times higher than that of an industrial grade.

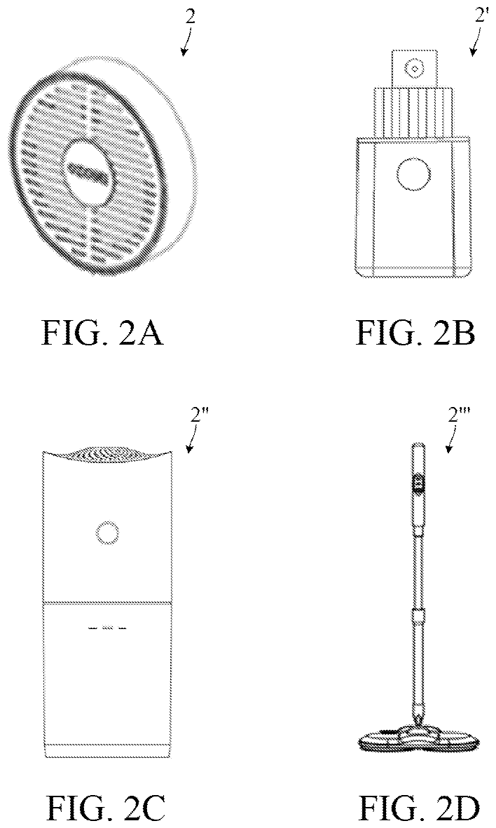

Please refer to FIG. 1 and FIG. 2 to FIG. 2D. FIG. 2A to FIG. 2D are schematic diagrams of 4 kinds of compact portable devices 2,2′,2″,2′″ for generating disinfectants. Besides the quality upgrade of materials for the fabrication of EO₃ anode, we also digitize, miniaturize and functionalize the new products of disinfectant generator, which includes a replacement for O₃ pen of FIG. 1. In FIG. 2, four compact and portable disinfectant generators are fabricated for sanitary applications. They are: (A) submissible-in-water generator of ozonated water or peroxone water in FIG. 2A, (B) hand-held sprayer for applying disinfectant on surfaces or objects to be sanitized in FIG. 2B, (C) automatic sprayer of disinfectant mist for humidification and sanitation in FIG. 2C, and (D) 3-in-one generator for sanitation-sweeping-mopping FIG. 2D. Only the device in FIG. 2A is operated in a container of static water, or under running water to generate infectants, which include O₃ or O₃ mixed with H₂O₂, depending on the cathode material employed. Device in FIG. 2A is also an advancing version of ozone pen, and the new device can yield disinfectants more than O₃. Devices in FIG. 2B, FIG. 2C and FIG. 2D are all equipped with a water compartment for electrodes to be plugged in the water to generate O₃ or O₃—H₂O₂, continuously or batchwise until the refilling of water. There are more ways of designing various generators to meet various needs.

No matter how the disinfectant generators are varied, the heart of the devices is always the anode and cathode. While the anode is Ni,Sb—SnO₂/Ti, the cathode can be AISI 304 or Co₃O₄-CNF/Ti. The next key component is the power source to drive the operation of electrodes. An advanced compact O₃ generator or O₃—H₂O₂ co-generator is configured in a form of 60 mm diameter disk. Through a standard USB (Universal Serial Bus) Type A Charging Cable, energy from a power source is charged the on-line storage to drive the devices to produce O₃, or O₃ mixed with H₂O₂, which depends on the cathode material employed therein. The disk devices can be submerged in a container of water to generate O₃ water or peroxone (H₂O₃) water for sanitation and disinfection at home or on the road.

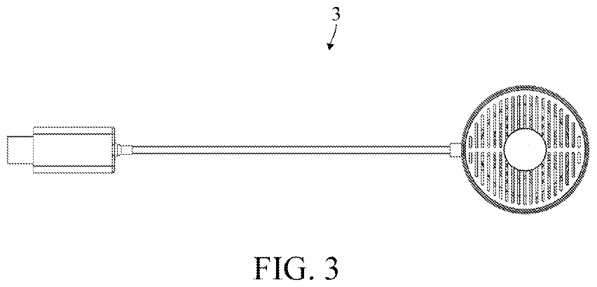

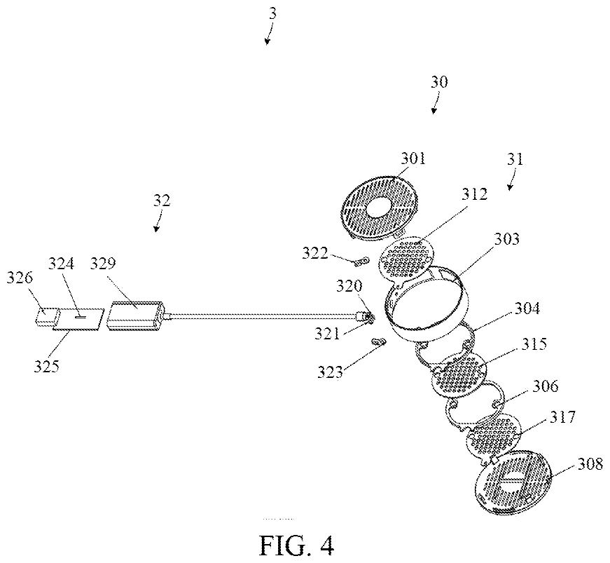

Please refer to FIG. 3 and FIG. 4. FIG. 3 is a schematic diagram of an embodiment of a compact generator 3 according to the present invention. FIG. 4 is an exploded-view drawing of the compact generator 3 in FIG. 3. In this embodiment, as shown in FIG. 3 and FIG. 4, the compact generator 3 includes a case body 30 and an electrolysis module 31. The case body 30 includes a top cover 301, a peripheral frame 303, a first separating spacer 304, a second separating spacer 306, and a bottom cover 308. The peripheral frame 303 defines a containing space of the compact generator 3 under the top cover 301 and the bottom cover 308. The electrolysis module 31 includes a first EO₃ cathode 312, an EO₃ anode 315 and a second EO₃ cathode 317. Furthermore. the compact generator 3 includes a power plug 32 with the charging cable. The power plug 32 includes a connector housing 329 with conducting cable, an anode conducting cable 320, a cathode conduction cable 321, a cathode connector 322 configured for linking the anode conducting cable 320 with two EO₃ cathodes 312,317, an anode connector 323 configured for linking the cathode conduction cable 321 with EO₃ anode 315, a LED indication light 324, a control circuit board 325, and a male connector 326 configured for plugging into a power source with USB plug socket. Moreover, the compact generator also can includes a hermetic seal between the top cover 301 and the bottom cover 308, so that the compact generator 3 become water proof.

There are 3 electrodes to form a basic unit of electrode set for O₃ generator, or (O₃+H₂O₂) generator, as shown in FIG. 3 and FIG. 4. The electrode set contains an anode sandwiched by two cathodes, wherein a ring spacer is placed between each of anode-cathode pair to define the electrode gap, as well as to prevent electrical short. While the anode is Sb,Ni—SnO₂/Ti, the cathode can be either 304 stainless steel or cobalt oxide plated on carbon nanofilm that is grown on Ti substrate (Co₃O₄-CNF/Ti). The latter cathode is composed of a thin layer of conductive carbon nanofilm (CNF) directly grown via 700-800° C. on Ti plate substrate for accepting cobalt (ii, iii) oxide [Co₃O₄] thereon. The said cobalt oxide is a catalyst that can catalyze the 2-electron reduction of O₂ into H₂O₂.

In one embodiment, the material of the first EO₃ cathode is SS304 and the material of the second EO₃ cathode is Co₃O₄-CNF/Ti in the compact generator 3. In another one embodiment, the materials of the first EO₃ cathode and the second EO₃ cathode are Co₃O₄-CNF/Ti.

By using Sb,Ni—SnO₂/Ti anode in conjunction with a SS304 cathode, the electrolysis of water will yield O₂/O₃ on anode and H₂ on cathode, respectively, at then individual potential as specified in Equation (2) and (3):

Anode reaction: 5H₂O→O₂↑+O₃↑+10H⁺+10e⁻ E°=1.60V (2)

Cathode reaction: 2H₂O+2e⁻→H₂↑+OH⁻ E°=0.0 V (3)

where E° is the gas evolution potential at standard state (0° C., 1 atm).

On the other hand, water electrolysis by coupling the same anode with Co₃O₄-CNF/Ti cathode will give a different cathode reaction as shown in Equation (4):

Cathode reaction: O₂+2H⁺+2e⁻→H₂O₂ E°=0.67V (4)

Furthermore, in the presence of O₃, H₂O₂ evolved will react with O₃ from anode as described in Equation (5):

2H₂O₂+2O₃→.OH+HO₂.+3O₂+H₂O (5)

In reaction (4), O₂ and H⁺ produced on anode will automatically diffuse to Co₃O₄ catalyst on cathode for reduction. Thus, O₂ evolution reaction (OER) on Sb,Ni—SnO₂/Ti anode and O₂ reduction reaction (ORR) on Co₃O₄-CNF/Ti cathode constitute a self-sustained system for the cogeneration of O₃ and H₂O₂ from a simple water electrolysis. Similar cogeneration of O₃ and H₂O₂ can also be seen in the report, by K Ishiwata et al. of Abstract #2655, in the 214th ECS meeting in year 2008 February. In their setup, the anode is β-PbO₂, and cathode is Ag marbles (Ag film deposited on Cu balls). The said anode and cathode electrodes are separated by an ion exchange membrane. Instead of using symmetrical electrodes as FIG. 3 and FIG. 4, Ishiwata's report employed anode and cathode in incompatible shape and dimensions. Furthermore, O₂ is supplied by flowing air into the cathodic compartment rather than on-line generation.

Two powerful disinfectants are formed in Equation (5). They are hydorxy radical (.OH) and hydroperoxyl or perhydroxyl radical (HO₂.), and they belong to reactive oxygen species (ROS) group. Using an unpaired electron, the said radicals can quickly extract one electron from bacteria, viruses and organic materials in water resulting in oxidation reaction of species being attacked, which will lead to the decomposition of the subjects. Ikai et al., in Antimicrobial Agents & Chemotherapy, p. 5086-5091, December (2010), presented a study of oral disinfection by photolysis of H₂O₂ by laser irradiation to form .OH for killing 4 species of pathogenic oral bacteria. In the presence of as low as 200-300 μM, or 3.4-5.1 ppm of .OH, a reduction of >99.999% of viable bacteria counts is attained within 3 minutes of treatment. Compared with other .OH generation systems, including, Fenton reaction, Haber-Weiss reaction, sonolysis of H₂O and photolysis of H₂O₂, the approach of this patent has the advantages of low-cost, convenience and environment friendly.

Online and in-situ production of a controllable dosages of O₃ and H₂O₂ is very beneficial to the applications of sanitation, disinfection, personal hygiene and wastewater treatment. Two novel materials are involved in the cogeneration of O₃—H₂O₂, one of them is Sb,Ni—SnO2/Ti anode that can consistently produce a constant source of pure O₂/O₃ at 2:1 yield ratio from water electrolysis at low power consumption. The other is Co₃O₄-CNF/Ti cathode containing nano carbon and Co(ii,iii) oxide that provide a synergistic catalysis on reducing O₂ to H₂O₂ through a 2-electron mechanism (Equation 4). On integrating Sb,Ni—SnO₂/Ti anode with Co₃O₄-CNF/Ti cathode for water electrolysis, O₃ and H₂O₂ can be quickly and simultaneously generated. But, the OER (oxygen evolution reaction) on anode and ORR (oxygen reduction reaction) on cathode are not equally made, they compete each other. Kinetically, O₂ evolution is faster than O₂ reduction or H₂O₂ formation. In order to enhance the throughput of H₂O₂, Co₃O₄-CNF can be directly grown on metal balls. Then, many Co₃O₄-CNF coated marbles can fill a conductive metal mesh bucket to form a packed cathode. Each ball in the bucket is a particle cathode that can concurrently reduce whatever quantity of O₂ gas provided by multiple anodes to H₂O₂. Consequently, the throughput of H₂O₂ can be scaled up for practical applications.

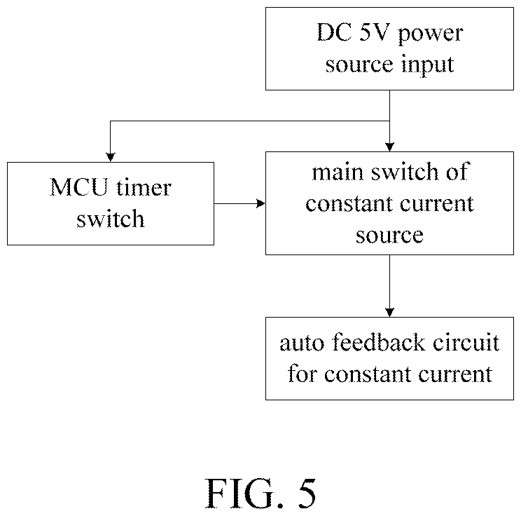

Please refer to FIG. 5. FIG. 5 is a logistic function blocks for providing a constant current for producing O₃ and H₂O₂. Apparently, water electrolysis requires power, and power consumption should be compensated by the values of materials produced. If the pay back is far less than the cost, then, the technology is meaningless. Based on the efficient anode and cathode, namely, Sb,Ni—SnO₂/Ti and Co₃O₄-CNF/Ti an efficient device that can conservatively provide power to the said electrodes to yield O₃—H₂O₂—H₂O₃ and their derives including .OH and HO₂. (Equation 5), is needed. This patent application chooses supercapacitor as the desired power device. For a high efficiency of energy utilization via supercapacitor, this patent also applies the following 2 platforms for operating the supercapacitor:

1) Charge-discharge Swing (CD Swing)

2) Reverse Charging

All three said items, the capacitor and 2 platforms, have been elaborated in one of LRS' prior applications, US2008/0181832. Only, a brief summary is provided for the 3 items in the current application.

### Supercapacitors

Same as battery, the supercapacitor is also an energy-storing device. Both energy devices are built based on 2 electrodes, that is, an anode and a cathode. Also, a gel polymer electrolyte (GPE) can be disposed between the electrodes of the two devices. However, the capacitor can be configured in a very different way from battery, which makes the former unique.

In supercapacitor, the GPE can impart 3 functions to the capacitor: as (a) separator, (b) conductor and (c) adhesive. This patent intends to use the GPE to bind many pieces of electrodes into a stack, which is called element of the capacitor. Multiple electrodes may be stacked vertically, or juxtaposedly, into a desirable height of element. Only the end electrodes of the stack, that is, top and bottom, is one side coated with an electrically active material, whereas the middle electrodes are all 2-side coated. Moreover, the middle electrodes are bipolar, which means two sides of an electrode can carry different polarities. The polarity of electrodes is determined by the connection of end electrodes. In other words, only the end electrodes are equipped with connectors for linking to an outer power source for charging. Once a stacked supercapacitor is charged, the end electrode that is hooked to the positive pole of the source will become anode, and the other end electrode is cathode. Through the effect of induction, the middle electrodes, or bipolar electrodes, will be polarized to + or − accordingly. In the next charging, the polarity of all electrodes can be reversed simultaneously through an electronically control. Electronically, the polarity of supercapacitor's electrodes can be reversed at any frequency at real-time as needed, which can be done by reversing the polarity of end electrodes via a controller at the desired frequency. The controller may be a central processing unit (CPU), micro processing unit (MPU), micro control unit (MCU). Frequency-guide Polarity Reversal (FPR) permits the bipolar-based supercapacitors to perform in direct current (DC) or alternating current (AC) mode. Moreover, the stacking of electrodes is an in-housing serial link (ISL) of multiple unit cells of supercapacitor. Each unit cell will add a cellular voltage to the total working voltage of module stacked. Hence, the higher the stack, the greater the working voltage of the module will be. Not only storing energy, the bipolar electrodes also work as serial linkage connectors. This feature permits the supercapacitor to be made into modules of high-voltage with high energy density, and high power density as well. However, the devices will have small dimensions and a low consumption of construction materials. It is an ultimate goal for this patent that the supercapacitor will be employed in all electrolytic generation of O₃ alone, or O₃ and H₂O₂ together.

### Charging-Discharging Swing (CD Swing) Platform

It is well known that the charging-discharging speed of all kind of capacitors are very fast. This is due to the capacitors employ only the surface of their electrodes for storing energy, thus, the charging of a shallow room can be completed in a very short time. Like proverbial saying “easy come easy go”, the low content of energy in the capacitors can be discharged quickly to an empty state. Only after recharging to its full energy state, the capacitors can perform again. Thus, the power provision of capacitors is intermittent, which is unacceptable to loads consuming large power constantly such as electric vehicles. This is the major hurdle for the developers of supercapacitor to overcome. How to make a non-stop supercapacitor that can perform as lithium battery on delivering 200-400 miles of range per charge? The answer contains many aspects of endeavors. This patent has presented the approach of effective enlargement of voltage by ISL (in-housing serial link) of bipolar electrodes. Since the energy and power of supercapacitor is proportional to the square of voltage. When the voltage is doubled, both the energy density and the power density of supercapacitor will be up quadrupled. Moreover, the bipolar electrodes also impart a dual, DC and AC, property to the bipolar-based supercapacitors via FPR (frequency-guide polarity reversal). It means that the FPR technique grants the supercapacitors to perform as either DC or AC devices. The said added-feature allows the supercapacitors to be charged wirelessly or remotely. Although supercapacitor can manage many types of energy use, it also requires operating platforms for upgrading its utilization efficiency of energy. Charging-discharging swing and reverse charging are designed as the platforms to meet the goal.

Under uncontrolled conditions, discharging may quickly drain the voltage of supercapacitors to a level below the driving voltage of loads, which nullifies the devices. For a high efficiency of energy utilization, supercapacitor should be operated by the effective energy only. Roughly, the energy under the first ⅓ portion of the rated voltage is regarded effective. In order to discharge and replenish just the effective energy, at least two groups of supercapacitor are needed in an operation of continuous provision of power. As the 1st group of supercapacitors has clone its set discharging, it will go into charging mode, and the 2nd group will immediately assume the discharging role. In the next cycle, the charging and discharging reciprocal sequence is repeated. The two capacitors continuously perform the reciprocal charging and discharging until the demand is fulfilled. Since the effective energy is only ⅓ portion of the total energy content of capacitors, so the discharging and recharging can be executed swiftly. The power delivery is not only consistent, but also the power level can be decided by adjusting the swing frequency, which can be executed easily using an IC controller.

### Reverse Charging Platform

During the discharging of supercapacitor modules made by ISL, it is observed that as the voltage of discharging side is fading, concurrently, the reverse side shows a building-up voltage in opposite polarity. The reverse charging is due to an energy harvesting on the return current from load via the supercapacitor back to the negative pole of the power source. Eventually, the “one side up and down the other” effect will impart the reverse side a higher value of volt in negative polarity than the number volt of the discharging side. On reversing the polarity of supercapacitor's leads by an IC controller, the exhausted super-capacitor can continuously discharge using the newly harvested energy. Thus, reverse charging may save energy from turning into heat, and it makes the supercapacitors based on bipolar electrodes unique and useful in many power applications.

Human beings live in a world filled with invisible microbes including viruses, pathogens and benign or infectious bacteria. They reside on the surface of fruits, vegetables, as well as seemingly fresh food and clean utensils. The microbial communities are ubiquitous, they are even identified to exist on human bodies. It is important to have an effective and affordable protecting means to stay healthy in our daily lives. The in-time provision of disinfectants, such as, the generators in this patent, will do the job.

### EXAMPLE

Ozone molecule (O₃) can only dissolve in water at 29.9 μg per 1 ml of water, 29.9 μg/ml or 29.9 ppm, at 20° C. water. If water temperature is lower, more O₃ can dissolve in water. When waterborne O₃ is above 1 ppm, it can liberate O₃ into air more than the safety levels (OSHA workplace maximum=0.1 ppm). The O₃ level produced by the generators of this patent can reach and beyond 1 ppm. However, the throughput of O₃ and the application of O₃ are adjustable and concealed. In the cogeneration type of generators, O₃ and H₂O₂ will form hydroxy radical (.OH) and hydroperoxyl radical (HO2.). The radicals not only are much more potent than O₃ on disinfection, also they inhibit O₃ leak from going above the allowed limit.

Straphylococcus aureus (SA) is one kind of gem that about 1 in 3 people may carry in their noses and skin. Most of the time, SA does not cause any harm. Similarly, Escherichia coli (E. coli) is another gem that lives in human body, that is, in the intestines. Most types of E.coli are harmless. Nevertheless, serious infection by SA or E. coli can be deadly. SA and E. coli are just two of the 12 most common pathogenic bacteria. For evaluating the disinfectants produced by the devices of this patent, we have outsourced the validation to a reputable biology lab using our disinfectants on eliminating the cultured E. coli and SA separately. The said bacterial were cultured on blood agar plates under 37° C. for 18 hours to the concentration levels of 5×10⁷-5×10⁸ CFU/ml. Then, the diluted bacteria solution was mixed with the O₃ water produced by the generator, at 100 μl bacteria liquid to 0.92 ml O₃—H₂O. In 10-minute reaction time, all tests showed that over 99.99% E.coli and SA were abated.

With the examples and explanations mentioned above, the features and spirits of the invention are hereinbefore well described. More importantly, the present invention is not limited to the embodiment described herein. Those skilled in the art will readily observe that numerous modifications and alterations of the device may be made while retaining the teachings of the invention. Accordingly, the above disclosure should be construed as limited only by the metes and bounds of the appended claims.

What is claimed is:

1. A compact generator for generating sterilizing materials, comprising:
a case body, comprising a containing space for containing water;
an electrolysis module, configured in the containing space for electrolyzing water to generate sterilizing materials, the electrolysis module further comprising:
an anode, configured to generate oxygen gas (O₂) and ozone gas (O₃) during electrolyzing water; and
at least one cathode, configured at one side of the anode, the cathode being configured to form hydrogen peroxide (H₂O₂) from the oxygen gas (O₂), generated by the anode, by reduction reaction;
wherein, the material of the anode is Sb,Ni—SnO₂/Ti, the material of the cathode is Co₃O₄-CNF/Ti wherein CNF is conductive carbon nanofilm, the ozone gas (O₃) and hydrogen peroxide (H₂O₂) are mixed in the containing space to form the sterilizing materials, the sterilizing materials comprises O₃, peroxone (H₂O₃), hydroxyl radical (.OH) and hydroperoxyl radical (HO₂.).

2. The compact generator of claim 1, wherein the anode consists essentially of tin oxide (SnO₂) formed by tin oxalate (SnC₂O₄).

3. The compact generator of claim 2, wherein the tin oxalate is formed by oxalic acid and Tin(II) salt, the Tin(II) salt is selected from the group of Dibutyltin dichloride, Dibutyltin maleate, Tin(II) acetate, Tin(II) bromide, Tin(II) chloride, Tin(II) 2-hexylhexanoate, Tin(II) fluoride, Tin(II) fluoroborate, Tin(II) iodide, Tin(II) pyrophosphate, Tin(II) sulfate and Tin(II) sulfide.

4. The compact generator of claim 1, wherein the at least one cathode comprises a first cathode and a second cathode, the anode is sandwiched by the first cathode and the second cathode.

5. The compact generator of claim 1, further comprising at least one supercapacitor and a power source, the supercapacitor being coupled to the electrolysis module and the power source, the supercapacitor being configured to discharge and store power from the power source and provide power to the electrolysis module for electrolyzing water.

6. The compact generator of claim 5, wherein the supercapacitor comprises a plurality of bipolar electrodes, and the bipolar electrodes are stacked in series.

7. The compact generator of claim 6, wherein the end bipolar electrode of the bipolar electrodes is connected to power source and configured to receive the polarity of the power source.

8. The compact generator of claim 5, wherein the supercapacitor has direct current (DC)/alternating current (AC) property by using the polarity reversal modulated by a controller.

9. The compact generator of claim 5, wherein the at least one supercapacitor comprises a first supercapacitor and a second supercapacitor, the first supercapacitor and the second supercapacitor are configured to deliver power continuously via charging and discharging swing.

10. The compact generator of claim 5, wherein the supercapacitor harvests power from return current from loads to the negative pole of the power source.

11. The compact generator of claim 5, further comprising a standard universal serial bus (USB) connector connected to the supercapacitor and the power source, the supercapacitor storing power through the standard USB connector.

12. The compact generator of claim 5, wherein the supercapacitor stores power by wireless charging.
